(19) United States
(12) Patent Application Publication
(10) Pub. No.: US 2022/0193403 A1
(43) Pub. Date: Jun. 23, 2022

(54) SYSTEM AND METHOD FOR WOUND HEALING

(71) Applicant: THE CLEVELAND CLINIC FOUNDTION, Cleveland, OH (US)

(72) Inventors: Francis A. Papay, Cleveland, OH (US); William Kolosi, Stow, OH (US)

(21) Appl. No.: 17/555,390

(22) Filed: Dec. 18, 2021

Related U.S. Application Data
(60) Provisional application No. 63/127,357, filed on Dec. 18, 2020.

(51) Int. Cl. A61N 1/30; A61M 1/00

(52) U.S. Cl. CPC A61N 1/303; A61M 1/915; A61M 1/962; A61M 2205/0283; A61N 1/44; A61M 2230/20; A61M 2205/3344; A61M 2205/3306; A61M 2230/65; A61M 2230/208

(57) ABSTRACT

A system and method is provided for healing a wound in a subject in need thereof. The system includes a wound dressing that has a therapeutic agent and at least one electrode in electrical communication with the therapeutic agent. The system also includes a power source in electrical communication with the at least one electrode and a vacuum source in fluid communication with the wound dressing.

### RELATED APPLICATIONS

The present application claims priority to U.S. Provisional No. 63/127,357 filed on Dec. 18, 2020 and which is incorporated by reference in its entirety.

### TECHNICAL FIELD

The present disclosure relates generally to a system and method for healing a wound, and more particularly to an iontophoretic, negative pressure system and method for delivering a therapeutic agent to a wound.

### BACKGROUND

Wounds are internal or external bodily injuries or lesions caused by physical means, such as mechanical, pressure, chemical viral, bacterial, or thermal means, which disrupt the normal continuity of structures. Such bodily injuries include contusions, wounds in which the skin is unbroken, incisions, wounds in which the skin is broken by a cutting instrument, and lacerations (e.g., wounds in which the skin is broken by a dull or blunt instrument). Wounds may also be caused by accidents or surgical procedures, in addition to pathologic conditions that cause cutaneous disruption.

When cells are injured or killed as a result of a wound, a wound healing step is desirable to resuscitate the injured cells and produce new cells to replace the dead cells. The healing process requires the reversal of cytotoxicity, the suppression of inflammation, and the stimulation of cellular viability and proliferation. Wounds require low levels of oxygen in the initial stages of healing to suppress oxidative damage, and higher levels of oxygen in the later stages of healing to promote collagen formation by fibroblasts.

One method used to promote the healing process is iontophoresis, which is a non-invasive technology for delivering nutrients, medicines, vitamins, minerals, therapeutic agents, drugs, or other bioactive agents using a small electric current, which causes an electrical field. In general, delivering such medicaments through iontophoresis involves applying an electromotive force that transports ions through the stratum corneum, the outermost layer of skin, and into the dermis, the inner layer of skin comprised of connective tissue, blood and lymph vessels, sweat glands, hair follicles, and an elaborate sensory nerve network. This same electromotive force can also transport ions through other subcutaneous tissue planes, wound granulation tissues, and biofilms.

Certain drawbacks exist for using iontophoresis to treat dermatological wounds, however. For example, treating wounds (e.g., dermatological wounds) using iontophoresis can cause localized pH alterations as a result of accumulation of electrolysis products and cellular necrosis. The build-up of such products can then shield bacteria, fungi, etc. in the region from penetration of therapeutic agents to the proper tissue depth.

### SUMMARY

According to one aspect, a system is provided for healing a wound of a subject in need thereof. The system comprises a wound dressing, a controller, and a fluid line for providing negative pressure wound therapy (NPWT) where the wound dressing includes a therapeutic agent and an electrode. In particular, the wound dressing has an upper layer comprising a porous material covered by an outer film defining a vacuum port. The wound dressing also comprises a lower tissue-contacting layer. A therapeutic agent and at least one electrode is disposed in the wound dressing, the at least one electrode being in electrical communication with the therapeutic agent. The controller is in electrical communication with the at least one electrode. The system further includes a fluid line having one end connected to the vacuum port and another end connected to a vacuum source. The vacuum source is configured to generate negative pressure within the porous material.

In another aspect, a system is provided for healing a wound of a subject in need thereof comprising a wound dressing, a controller, and a fluid line for providing NPWT where the wound dressing include at least one capacitor. In particular, the wound dressing has an upper layer comprising a porous material covered by an outer film defining a vacuum port. The wound dressing also includes a lower tissue-contacting layer. A therapeutic agent and at least one capacitor is disposed in the wound dressing, the at least one capacitor being in electrical communication with the therapeutic agent. The controller is in electrical communication with the at least one capacitor. The system further includes a fluid line having one end connected to the vacuum port and another end connected to a vacuum source. The vacuum source is configured to generate negative pressure within the porous material.

In another aspect, a system is provided for healing a wound of a subject in need thereof comprising a wound dressing, a controller, and a fluid line for providing NPWT, where the wound dressing include polymer foam layers that collectively serve as a capacitor. In particular, the wound dressing has an upper layer, a tissue-contacting layer, and an intermediate layer. The upper layer comprises a porous material covered by an outer film defining a vacuum port. The intermediate layer comprises a first and a second segment of electrically conductive polymer foam separated by a segment of dielectric form, the first and second segments having opposing polarities. A therapeutic agent is disposed in the wound dressing. The controller is in electrical communication with the intermediate layer. The system further includes a fluid line having one end connected to the vacuum port and another end connected to a vacuum. The vacuum is configured to generate negative pressure within the porous material.

In another aspect, a method of healing a wound in a subject in need thereof is provided. The method comprises placing at least a portion of a wound dressing on a wound of a subject. The wound dressing comprises a porous material covered by an outer film defining a vacuum port. The method further includes applying an electrical field to the wound to enhance migration of cells into the wound to regenerate wound tissue. The method also includes removing fluid or debris from the wound by applying negative pressure to the wound. The applying and removing steps can be repeated until the wound is sufficiently healed.

In another aspect, a method of healing a wound in a subject is provided where a wound dressing comprising a therapeutic agent and an electrode is utilized. In particular, the method comprises placing at least a portion of wound dressing on a wound of the subject. The wound dressing has an upper layer comprising a porous material covered by an outer film defining a vacuum port. The wound dressing also has a lower tissue-contacting layer. A therapeutic agent and at least one electrode are disposed in the wound dressing, the at least one electrode being in electrical communication with the therapeutic agent. The method includes activating the at least one electrode to generate an electrical field to motivate the therapeutic agent into the wound or tissue surrounding the wound. The method further comprises removing fluid or debris from the wound by applying negative pressure to the wound. The activating and removing steps can be repeated until the wound is sufficiently healed.

### BRIEF DESCRIPTION OF THE DRAWINGS

FIG. 1 is a block diagram of a system for healing a wound in a subject according to an embodiment of the present disclosure.

FIG. 2 is a cross-sectional view of a wound dressing of a system for healing a wound in a subject according to an embodiment of the present disclosure.

FIG. 3 in a perspective view of a micro-needle electrode array of a wound dressing according to an embodiment of the present disclosure.

FIG. 4 is a block diagram of a system for healing a wound in a subject in need thereof according to an embodiment of the present disclosure.

FIG. 5 is a block diagram of a system for healing a wound in a subject in need thereof according to an embodiment of the present disclosure.

FIG. 6 is a cross-sectional view of a system for healing a wound in a subject according to an embodiment of the present disclosure showing therapeutic agents being delivered into a wound and removal of fluid and/or debris from the wound (indicated by the arrows).

FIG. 7 is flow diagram of methods of healing in wound in a subject in need thereof according to an embodiment of the present disclosure.

FIG. 8 depicts components of a system for healing a wound in a subject in need thereof according to an embodiment of the present disclosure.

FIG. 9 is a schematic illustration of a system for healing a wound in a subject in need thereof according to an embodiment of the present invention.

FIG. 10 is a schematic illustration of a system for healing a wound in a subject in need thereof according to an embodiment of the present invention.

### DETAILED DESCRIPTION

As used herein with respect to a described element, the terms “a,” “an,” and “the” include at least one or more of the described element including combinations thereof unless otherwise indicated. Further, the terms “or” and “and” refer to “and/or” and combinations thereof unless otherwise indicated. It will be understood that when an element is referred to as being “over,” “on,” “attached” to, “connected” to, “coupled” with, “contacting,” “in communication with,” etc., another element, it can be directly over, on, attached to, connected to, coupled with, contacting, or in communication with the other element or intervening elements may also be present. In contrast, when an element is referred to as being “directly over,” “directly on,” “directly attached” to, “directly connected” to, “directly coupled” with, “directly contacting,” or in “direct communication” with another element, there are no intervening elements present. An element that is disposed “adjacent” another element may have portions that overlap or underlie the adjacent element. By “substantially” is meant that the shape, configuration, or orientation of the element need not have the mathematically exact described shape, configuration or orientation but can have a shape, configuration or orientation that is recognizable by one skilled in the art as generally or approximately having the described shape, configuration, or orientation. The terms “horizontal,” “vertical,” “left” and “right” refer to the orientation of components as they are depicted in the figure. The term “subject” includes a human being.

As used herein, the terms “therapeutic agent”, “drug”, “agent”, “chemical compound”, and “chemical substance” can refer to any therapeutically effective molecule or moiety (i.e., molecules or moieties that are capable of having a biological effect), such as pharmaceutical agents, drugs, or biological agents.

As used herein, the term “wound” can refer to damage or loss to any one or combination of skin layers caused by cuts, incisions (including surgical incisions), abrasions, microbial infections, diseases or disorders, necrotic lesions, lacerations, fractures, contusions, burns and amputations. Non-limiting examples of wounds can include bed sores, thin dermis, bullous skin disease, and other cutaneous pathologies, such as subcutaneous exposed wounds that extend below the skin into the subcutaneous tissue. In some instances, a subcutaneous exposed wound may not affect underlying bones or organs. In certain embodiments, the wound is not located in the eye or ear.

As used herein, the terms “treatment” and “treating” can refer to obtaining a desired physiologic, dermatological, or cosmetic effect and improving the patient's condition compared to the patient's condition prior to treatment. The effect may be prophylactic in terms of completely or partially preventing a disease, disorder, or symptom thereof and/or may be therapeutic in terms of a partial or complete cure for a disease, disorder, and/or symptom attributable to the disease or disorder. Thus, the terms can cover any treatment of a disorder or disease in a subject, such as: (a) preventing a wound from occurring in a subject that may be predisposed to developing the wound but has not yet been diagnosed as having it; (b) inhibiting a wound, e.g., arresting its development; or (c) relieving, alleviating, or ameliorating a wound by, for example, causing regression of the wound.

As used herein, the term “cosmetic effect” can refer to any treatment by the present disclosure that preserves, restores, bestows, simulates, or enhances the appearance of bodily beauty or appears to enhance the beauty or youthfulness, specifically as it relates to the appearance of tissue or skin.

As used herein, the terms “healing” and “heal” can refer to improving the natural cellular processes and humoral substances of tissue repair such that healing is faster, and/or the resulting healed area has less scaring, and/or the wounded area possesses tissue strength that is closer to that of uninjured tissue, and/or the wounded tissue attains some degree of functional recovery. The terms can additionally or alternatively refer to the physiological process wherein a wounded area returns to an effectively normal state. When the wound is an open wound, for example, healing can refer to the process whereby the skin or mucosa re-forms a continuous barrier. The skilled artisan will appreciate that, after healing, the area of the wound may comprise scar tissue that is not identical to the surrounding tissue.

As used herein, the term “operably connected” can refer to a connection between components or entities whereby the one entity is in some way attached to a second entity. An operable connection can be directly between first and the second entities, for example, through the use of threaded fasteners, plastic or metallic tube fittings, nails, chemical adhesives, tape, weldment, or the like. A direct connection between first and the second entities can be non-detachable, for example, through the use of chemical adhesives or weldment, or detachable, for example, through the use of removable fasteners, such as threaded fasteners. Alternatively, an operable connection can be indirectly between first and the second entities via one or more intermediate entities.

As used herein, the term “fluid communication” can refer to two chambers, vessels, tanks, or other structures containing a fluid, such as a liquid or gas, where the fluid-containing structures are directly or indirectly connected together (e.g., by a line, pipe or tubing) so that a fluid can flow between the two fluid-containing structures. Therefore, two chambers that are in “fluid communication” can, for example, be connected together by a line between the two chambers, such that a fluid can flow freely between the two chambers.

As used herein, the term “electrical communication” can refer to the ability of a generated electric field to be transferred to, or have an effect on, one or more components of the present disclosure. In some instances, the generated electric field can be directly transferred to a component (e.g., via a wire or lead). In other instances, the generated electric field can be wirelessly transferred to a component.

The present disclosure generally relates to a system and method for healing a wound (e.g., a dermatological or subcutaneous exposed wound), and more particularly to an iontophoretic, negative-pressure system and method for delivering at least one therapeutic agent to a wound. Such a system can be used to seamlessly control drug flow patterns in and about a wound while also removing unwanted materials from the wound site, such as bacteria and other debris. In doing so, the present disclosure maximizes tissue perfusion and the interaction between therapeutic agents and wound tissue. Although the present disclosure is described below in terms of treating decubital ulcers or “bed sores”, it will be appreciated that other types of wounds can be treated by the present disclosure.

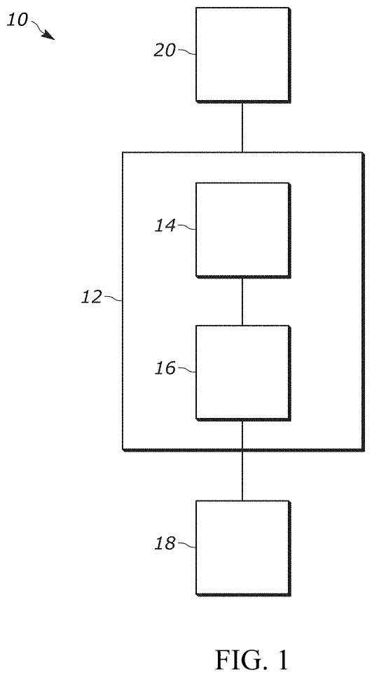

One aspect of the present disclosure relates to a system for healing a wound in a subject. Referring to FIG. 1, in an embodiment, system 10 comprises a wound dressing 12 having a therapeutic agent 14 and at least one electrode or at least one capacitor (also referred to herein as “electrode” or “capacitor”) 16 in electrical communication with therapeutic agent 14. System 10 also includes a controller 18 in electrical communication with electrode 16 and a vacuum 20 in fluid communication with wound dressing 12 to provide negative pressure wound therapy (NPWT) to the wound. The wound dressing can be fabricated from a material to conformally contact the skin of a subject. The wound dressing can also have a flattened or planar configuration for flush placement against the subject's skin.

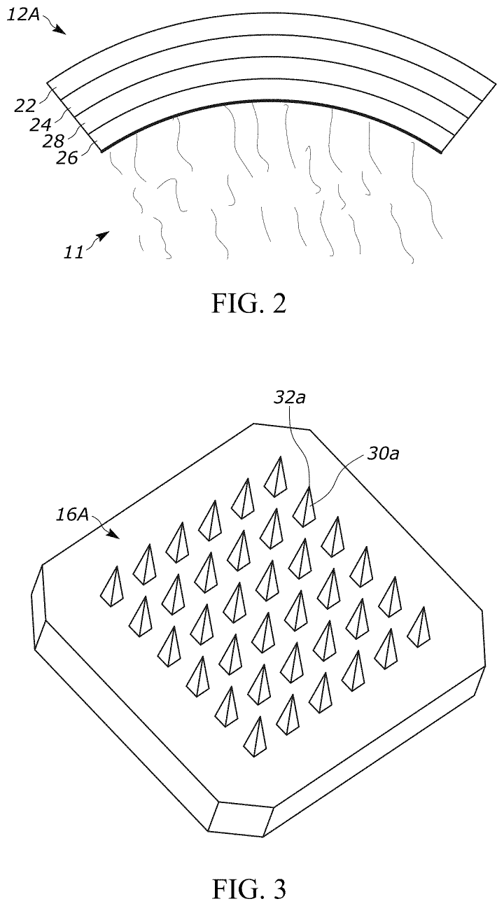

Referring to FIG. 2, a wound dressing 12A can comprise an outer film 22, an upper layer 24 below outer film 22, a lower tissue-contacting layer 26 and an intermediate layer 28 between upper layer 24 and lower tissue-contacting layer 26. Although the layers of wound dressing 12A are shown to be contiguous with adjacent overlying layers, other layers and components can also be present between the outer film and the layers depicted in FIG. 2. Along the same lines, the wound dressing can include fewer layers than those depicted in FIG. 2 so long as the wound dressing includes at least one layer for placement of electrode 16 (or a capacitor as described in more detail below) and therapeutic agent 14. The layers can have different thicknesses, densities and porosities (in embodiments where a layer is porous). One or more layers can be adjacent to another layer in a horizontal, left/right fashion as opposed to a vertical, stacked fashion as illustrated in FIG. 2. Although wound dressing 12A is depicted in FIG. 2 as having distinct layers, the wound dressing can have a more homogenous configuration.

In certain embodiments, at least a portion of layers 24, 26 and/or 28 are porous to facilitate movement of a therapeutic agent and debris and/or fluid to and from wound 11 or tissue surrounding wound 11. The layers of the wound dressing can comprise a matrix formed of sponge or gel (e.g. a hydrogel), a foam, mesh or expandable material. The dimensions of the pores of the layers can be the less than, the same as, or greater than the dimensions of the pores of other layers of the wound dressing. In embodiments, where the wound dressing has an upper layer comprising a porous material covered by an outer film, the pore size of the porous material (preferably greater than 10 microns) and inter-pore connectivity permits negative pressure to draw wound exudates, dead tissue, biofilm, etc. into the porous material. As such, the material(s) used to form a porous layer of the wound dressing can include materials that permit the flow of a fluid and/or debris from a wound. For example, a porous layer can comprise a biocompatible, non-biodegradable polymeric material made from a homopolymer, a copolymer, straight polymers, branched polymers, cross-linked polymers, stimuli-responsive polymers, or a combination thereof. Examples of such polymers can include silicone, polyvinyl alcohol, ethylene vinyl acetate, polylactic acid, nylon, polypropylene, polycarbonate, cellulose, cellulose acetate, polyglycolic acid, polylactic-glycolic acid, cellulose esters, polyethersulfone, acrylics, their derivatives, or combinations thereof.

In certain embodiments, therapeutic agent 14 is disposed in lower tissue-contacting layer 26 and at least one electrode 16 is disposed in a layer or the outer film 22 above tissue-contacting layer 26. For example, at least one electrode 16 can include a first electrode and a second electrode having opposing polarities. The first electrode can be disposed in outer film 22 and the second electrode can be disposed under outer film 22 and above tissue-contacting layer 26, such as in upper layer 24 or intermediate layer 28 In other embodiments, both the first and second electrode can be disposed in layers below outer film 22 and above the wound tissue 11, such as in upper layer 24 or intermediate layer 28. In yet other embodiments, intermediate layer 28 can comprise a dielectric barrier or two interdigitated electrodes.

In addition to being in different locations within the wound dressing, the electrode can be constructed from different materials or have different configurations. For example, the electrode can be constructed from electrically conductive materials or coatings. In certain embodiments, the electrode can comprise electrically conductive polymer foam or an electroactive polymer that expands when an electrical stimulus is applied thereto. Referring to FIG. 3, electrode 16B can be an array of micro-needles 30 electrically connected to each other to form, collectively, a single electrode. Micro-needles 30 can penetrate wound tissue, such as biofilm, that forms over a wound. Only the ends 32 of micro-needles 30 can be the electrical contact for purposes of moving an electric field deeper into the wound. In certain embodiments, where the at least one electrode comprises a first and a second electrode having opposing polarities, the first electrode can be micro-needles disposed in a tissue-contacting layer of the wound dressing and the second electrode can be disposed in a layer above the tissue-contacting layer, such as the outer film. Micro-needles can also be used to deliver a liquid solution into biofilm through the internal lumens of the micro-needles.

The electrode can comprise any one or combination of electrodes capable of providing an electric field sufficient to motivate a therapeutic agent into a wound and/or tissue surrounding the wound. To ensure proper transmission of electrical energy, the electrode can include at least two separate, electrically-conductive portions or components that are biased against one another. The electrode can be made from flexible, electrically-conductive materials that are capable of conducting an electric current. For example, materials used to form the electrode can include metals or metal alloys, such as platinum, platinum-iridium, stainless steel, gold, copper, gold-plated copper, zinc or the like. Additionally or optionally, at least a portion of the electrode can be embedded within a polymeric material (or other similar material) (e.g., silicone) to protect wound tissue from abrasion and promote biocompatibility and/or electrical conduction.

The electrode can comprise a regularly-shaped, irregularly-shaped, uniform, and/or non-uniform electrode capable of providing a uniform or non-uniform electric field sufficient to motivate any polarizable chemical compound, including compounds that are difficult to polarize, such as non-polar drugs and large molecule compositions into a wound and/or tissue surrounding a wound. In one example, the electrode can comprise a first electrically-conductive material (e.g., a treatment electrode) and a second electrically-conductive material (e.g., a return electrode) that are adapted for motivating or causing the migration of ionizable therapeutic molecules via iontophoresis. In another example, the electrode can comprise an interdigitated electrode capable of providing a non-uniform electric field to an area sufficient to induce dielectrophoretic transport of a therapeutic agent.

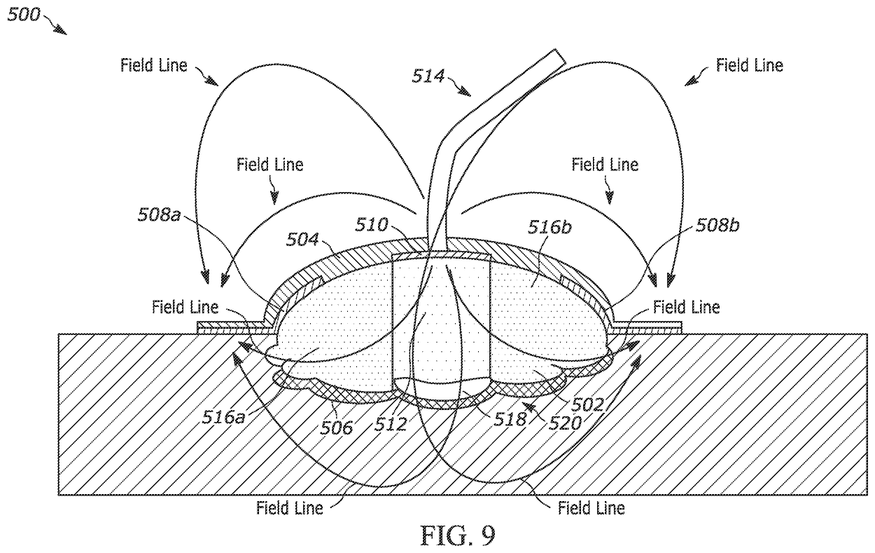

In alternative embodiments, a system for healing a wound of a subject in need thereof comprises a wound dressing including a capacitor. For example, a wound dressing can include an upper layer comprising a porous material covered by an outer film defining a vacuum port and can also include a lower tissue-contacting layer. A therapeutic agent can be disposed in the wound dressing as well as at least one capacitor in electrical communication with the therapeutic agent. For example, referring to FIG. 9, a wound dressing 500 can include an intermediate layer 502 between the upper layer 504 and the lower layer 506. This intermediate layer can comprise a first and a second segment of electrically conductive polymer foam or other form of an electrode 508 and 510 respectively separated by a segment of dielectric foam 512. The first and second segments have opposing polarities. The three foam segments together can create a capacitor, the capacitor having adjacent non-conductive foam 516. There may be multiple such capacitors assembled into a single porous layer. The control of these capacitors can be such that each capacitor goes through a charge and discharge cycle. There can be a sequence of charge and discharge cycles (indicated by the field lines) that can be tuned to optimize the healing effects of an electrical field, as well as delivery of a therapeutic agent 518 to a wound 520. Such a system also includes a power source (not shown) in electrical communication with the at least one capacitor in addition to a fluid line having one end connected to the vacuum port/drain 514 and another end connected to a vacuum, the vacuum configured to generate negative pressure within the porous material.

The therapeutic agent of the wound dressing can be in ionic form, such as a stabilized ionic therapeutic agent. Non-limiting examples of therapeutic agents include a stabilized ionic silver such as silver dihydrogen citrate. In certain embodiments, the therapeutic agent can comprise two components with the first component disposed in a layer or portion of wound dressing that also includes the electrode. The second component of the therapeutic agent can assume a liquid form that is delivered separately. When the two components of the therapeutic agent come in contact with each other, a chemical reaction can occur resulting in an ionic form of the therapeutic agent that can be delivered into the wound by the electric field generated by the electrode. Alternatively, the therapeutic agent can comprise two or more molecules or drug compounds that are disposed in the lower tissue-contacting layer and only when an electrical field is applied, do the two molecules/compounds react, forming an ionized form of the drug that can consequently be delivered to the wound via the electrical field. The therapeutic agent can be in liquid or solid form and can be molecules or drug compounds. The therapeutic agent can be disposed in a hydrogel or foam material and/or encapsulated in micelles, liposomes, microspheres, or nanoparticles. The therapeutic agent can be disposed or integrated into a coating or portions of the wound dressing can be doped, impregnated or saturated with the therapeutic agent.

The type of therapeutic agent in the wound dressing can be selected based upon the particular type of wound being treated. Exemplary therapeutic agents include dermatological agents, antibacterial agents, antifungal agents, anticonvulsant agents, antihypertensive agents, anticancer agents, immunomodulatory agents, antiviral agents, anesthetics, analgesics, tranquilizers, sedatives, muscle relaxants, non-steroidal anti-inflammatory agents, cosmetic agents, biologics, small molecules, polynucleotides, polypeptides and steroids. Other examples of therapeutic agents, such as those that can be used for cosmetic or aesthetic purposes, include botox, botulinum toxin, hyaluronic acid, collagen and elastin.

More specific examples of therapeutic agents can include vitamin A, C, D or E, alpha-hydroxy acids, such as pyruvic, lactic or glycolic acids, beta-hydroxy acids, caffeine, theobromine, lanolin, vaseline, aloe vera, methyl or propyl parban, pigments, dyes and the like for tattooing and make-up effects, estrogen, make-up agents, anti-aging agents, pigments, such as iron oxide and titanium oxide for use after dermabrading for tattoo removal, iodine to reduce scar tissue, nutrients, DNA, RNA, corticosteroids and -caine-type compounds, such as lidocaine in base form, estradiol, progesterone, demegestone, promegestone, testosterone and their esters, nitro-compounds, such as nitroglycerine and isosorbide nitrates, nicotine, chlorpheniramine, terfenadine, triprolidine, hydrocortisone, oxicam derivatives, such as piroxicam, ketoprofen, mucopolysaccharides, such as thiomucase, buprenorphine, fentanyl and its analogs, naloxone, codeine, dihydroergotamine, pizotiline, salbutamol, terbutaline, prostaglandins, such as misprostol and emprostil, omeprazole, imipramine, benzamides, such as metaclopramide, scopolamine, peptides, such as growth releasing factor, epidermal growth factor and somatostatin, cloidine, dihydroxypyridines, such as nifedipine, verapamil, ephedrine, proanolol, metoprolol, spironolactone, thiazides, such as hydrochlorothiazide, flunarizine, syndone imines, such as molsiodmine, sulfated polysaccharides, such as heparin fractions, and salts of such compounds with physiologically acceptable acids and bases.

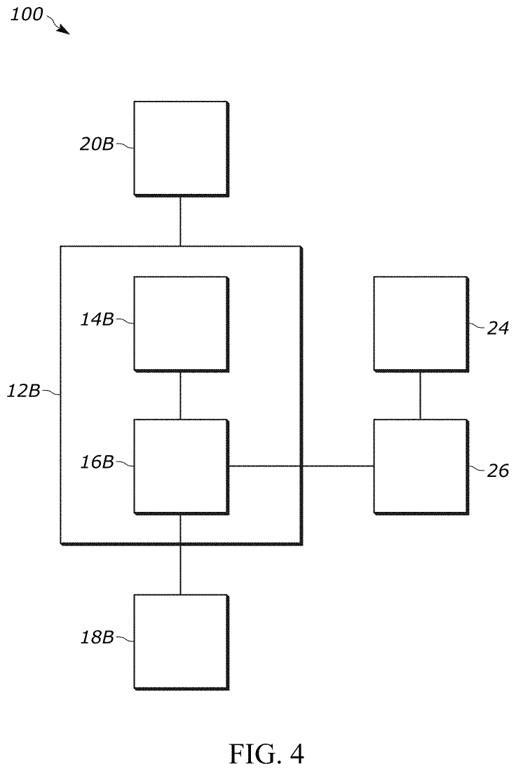

In certain aspects, the present disclosure provides systems that include feedback mechanisms that can be used to heal a wound of a subject in need thereof. Referring to FIG. 4, in an embodiment, a system 100 includes a wound dressing 12B including therapeutic agent 14B and electrode 16B in electrical communication with therapeutic agent 14B. System 100 also includes a vacuum 20B in communication with wound dressing 12B. System also includes a sensor 24 configured to sense a physiological parameter associated with the wound and generate a sensor signal based on the physiological parameter. In this aspect, a controller 18B is in electrical communication with electrode 16B and sensor 24. Controller 18B is configured to provide power to electrode 16B and is programmed to generate a drive signal to automatically control activation of electrode 16B or parameters of the electrical field generated by electrode 16B in response to the sensor signal to heal the subject's wound. In other words, feedback systems that can be used to heal a wound include a sensor that can be in communication with a control system that dynamically adjusts output signals to the electrode in order to optimize the electrical field and iontophoretic drug delivery. The sensor can be disposed in the wound dressing or can be configured for placement on another site of the patient's body. The sensor can be, for example, a temperature, pH, blood oxygen saturation level, light, or impedance sensor. In terms of a light sensor, certain bacteria autofluoresce when exposed to certain wavelengths of light. Thus, in embodiments where a system includes a wound dressing containing or in communication with a light emitter (described in more detail below), a light sensor can detect the presence of such bacteria.

Two or more components of the system can be in wireless communication with one another. In other instances, two or more components of the system can be in wired communication with one another. It will be appreciated that some components of the system can be in wireless communication with one another while other components are in wired communication with one another.

The controller is configured and programmed to control activation and iontophoresis parameters of the electrode(s). The controller can include software programmed to generate a drive signal to control activation of the electrode(s) and stimulation or parameters delivered by the electrode. For example, the controller can control the pulse waveform, the signal pulse width, the signal pulse frequency, the signal pulse phase, the signal pulse polarity, the signal pulse amplitude, the signal pulse intensity, the signal pulse duration and combinations thereof of an electrical signal delivered by the electrode.

The controller can include, for example, one or more microprocessors under the control of a suitable software program. The controller can be configured to record and store data indicative of physiological parameters associated with the wound in a patient. Therefore, the controller can generate a drive signal to indicate delivery or adjustment of delivery of an electrical signal by the electrode device when a physiological parameter increases or decreases above a certain threshold value (or range of values), for example, such as a normal or baseline level.

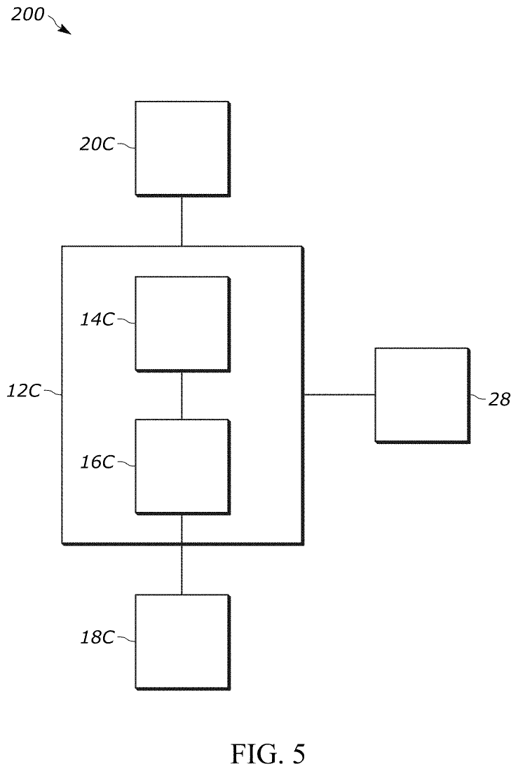

In certain aspects, the present disclosure provides systems that include a physical emitter that can be used to heal a wound of a subject in need thereof. Referring to FIG. 5, in an embodiment, a system 200 includes a wound dressing 12C, a controller 18C, a vacuum 20C and a physical emitter 28. The physical emitter can be disposed in the wound dressing or can be an external component in communication with the wound dressing. The physical emitter can be, for example, a sound/acoustic emitter, a shock wave emitter, a light emitter, or a cold plasma emitter. A sound emitter includes an ultrasound emitter, such as a piezoelectric material, and can emit sound waves, such as ultrasound waves, within the subject's wound to disrupt biofilm. Similarly, a shock wave emitter can emit shock waves to disrupt biofilm. A light emitter can emit light having an anti-microbial effect. For example, the light emitter can emit white light, blue light, or ultraviolet light to kill bacteria or disrupt the growth of bacteria within the wound. A cold plasma emitter can have a similar effect. A further benefit of cold plasma is that as a byproduct, it releases nitric oxide, which can contribute to vasodilation to enhance wound healing. The physical emitter can also activate otherwise dormant sensors disposed in the wound dressing.

In certain embodiments, an electroactive polymer is incorporated into the wound dressing such that application of an electrical field causes the electroactive polymer to expand and consequently the wound dressing expands to fill the wound cavity.

Systems as described herein include a vacuum that is in fluid communication with the wound dressing to provide NPWT. NPWT is a therapeutic technique using a vacuum to promote healing in acute or chronic wounds and enhance healing of first and second degree burns for example. The therapy generally involves controlled application of sub-atmospheric pressure to a local wound environment using a sealed wound dressing connected to a vacuum pump or other vacuum source. The continued vacuum draws out fluid from the wound to clean and drain the wound bed and increases blood flow to the area. The vacuum can be applied continuously or intermittently, depending on the type of wound being treated and the clinical objectives. The wound dressing can also be periodically changed. NPWT devices can allow delivery of fluids, such as saline or antibiotics to irrigate the wound. A wound dressing having a porous material sealed to an outer film can be fitted to the contours of a wound (which can be covered with a non-adherent dressing film). A drainage tube can be connected to the wound dressing (such as an outer film of the wound dressing) through an opening, such as a vacuum port of the outer film. A vacuum tube can be connected through an opening in an film drape to a canister on the side of a vacuum, such as a pump, turning an open wound into a controlled, closed wound while removing excess fluid from the wound bed to enhance circulation and remove wound fluids.

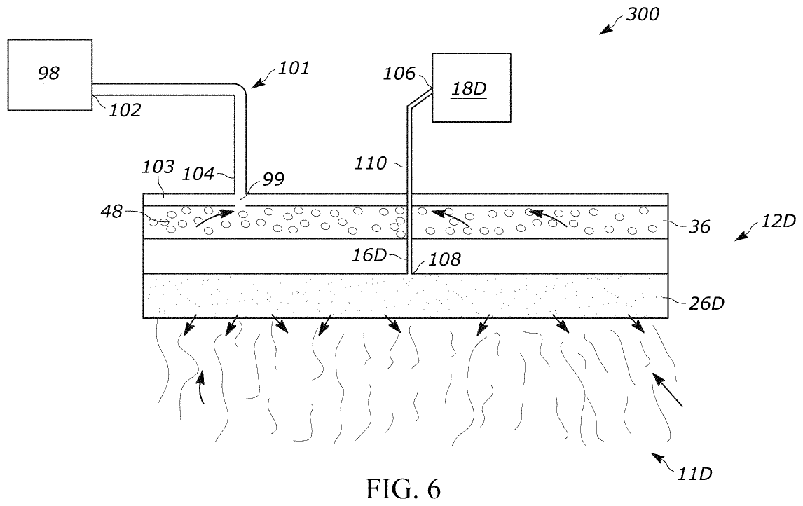

Referring to FIG. 6, vacuum 98 of system 300 can be in fluid communication with wound dressing 12D via fluid line 101 (e.g., medical-grade tubing). For example, a first end 102 of fluid line 101 is securely connected to vacuum source 98, while a second end 104 of fluid line 101 is securely connected to a vacuum port (opening) 99 of outer film 103 of the wound dressing 12B. As shown in FIG. 6, second end 104 of fluid line 101 extends through an upper portion or layer 36 of wound dressing 12D so that a lumen of fluid line 101 is in fluid communication with the porous material 48 of upper portion or layer 36. Second end 104 of fluid line 101 can be affixed to the vacuum port of the wound dressing 12D via any suitable method, such as gluing, stapling, stitching, different forms of mechanical connection, etc. Vacuum 98 can include any suitable device or apparatus capable of generating or providing negative pressure within a portion of wound dressing 12D. For example, vacuum 98 can comprise a pump. The vacuum can be operated at or below atmospheric pressure (i.e., negative pressure), and can be applied constantly, periodically, or cyclically. As described in more detail below, the vacuum can promote wound healing by removing fluid and/or debris that accumulates at or within the wound.

Systems as described herein include a controller that is configured to be a power source that is in electrical communication with an electrode and is capable of delivering an electrical signal to the electrode. The power source also can provide electrical power to other components of the system that require it. The power source can be configured to provide an AC signal, a DC signal, or a combination thereof. In some instances, the power source is configured to provide a signal having certain characteristics. As described below, the certain characteristics can include an orienting frequency or a motivating frequency. The power source can be electrically connected to the electrode via a direct electrical link or a wireless link (e.g., an RF link). As shown in FIG. 6, for example, proximal and distal ends 106 and 108 of an electrical lead 110 can be electrically connected to controller 18D and electrode 16D, respectively. The power source of the controller can be any of the following, individually or in combination: wireless power, battery power, and charge banks. When the power source includes wireless power, it can be configured to receive power from a remote transducer via wireless power transfer technologies, such as inductive coupling, resonate inductive coupling, capacitive coupling, near-field coupling, mid-field coupling, far-field coupling, microwave power, ultrasonic/acoustic power, and light power. When the power source includes battery power, the batteries can be disposable batteries, such as nickel-cadmium batteries or rechargeable batteries, such as lithium-ion batteries. When power source includes charge banks, the charge banks can include capacitors, inductors, and super-capacitors, which can be used on a standalone basis or in combination with the battery and/or wireless power.

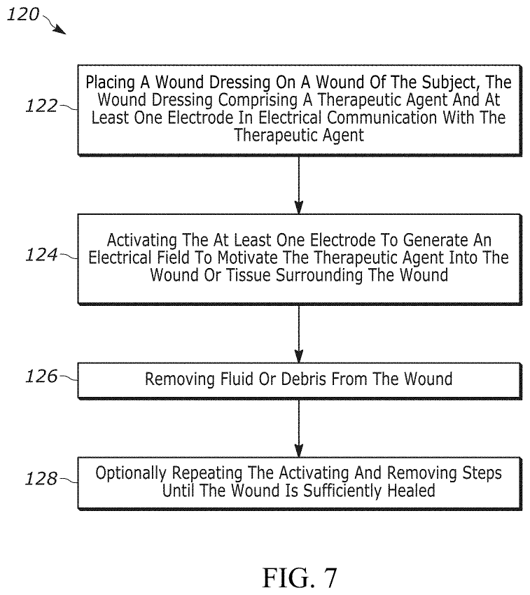

FIG. 7 is a process flow diagram illustrating a method 120 for healing a wound in a subject in need thereof with reference to an exemplary system 300 depicted in FIG. 6. At step 122, method 120 includes placing a wound dressing or a portion thereof on wound of a subject. The wound dressing can comprise a therapeutic agent and at least one electrode in electrical communication with the therapeutic agent. The particular placement location, type of therapeutic agent, and the size and shape of the wound dressing can depend upon the type of wound being treated (e.g., deep or shallow, acute or non-acute, etc.), the location of the wound, the subject's age, any underlying disease or condition, as well as other factors. For example, application of a system to a wound can be done as soon as possible following an acute injury. Depending upon the type of injury, however, application of a system to the wound may be initiated any time after injury or whenever deemed medically necessary. Before placement of the wound dressing at step 122, cleaning and debridement of the wound may be needed.

In embodiments where the wound dressing includes a lower tissue-contacting layer, such a layer can be placed into contact with the wound so that the lower tissue-contacting layer partially or completely covers the wound. Where the subject is suffering from a bed sore, for example, the entire surface of the bed sore can be covered by the lower tissue-contacting layer.

If it has not been done so already, the vacuum and controller can be operably connected with the wound dressing or components thereof, such as the electrode. After doing so, method 120 comprises activating the electrode, via the power source, to generate an electrical field to motivate the therapeutic agent (in lower-tissue contacting layer 26D, for example, as depicted in FIG. 6) into the wound or tissue surrounding the wound (step 124) as schematically illustrated by the arrows pointing towards wound 11D in FIG. 6. Activation of the controller provides one or more electrical signals to the electrode, which polarizes the therapeutic agent and causes the polarized therapeutic agent to be motivated into the wound and/or the tissue surrounding the wound via electromotive forces. The characteristics of the electrical signal (e.g., frequency, voltage, etc.) can be varied as needed, as long as migration of the therapeutic agent is aided by the electromotive force. The electrode can be activated continuously or periodically to control the desired rate and frequency of delivery of the therapeutic agent into the wound.

In one example, the therapeutic agent can be motivated into the wound and/or the tissue surrounding the wound via iontophoresis. To deliver the therapeutic agent via iontophoresis, the electrode of the system can comprise a treatment electrode and a return electrode. Additionally, the controller can include a low-voltage, DC or AC signal generator having positive and negative terminals (not shown) that are in electrical communication with the treatment electrode and the return electrode, respectively. Activation of the controller will cause the treatment and return electrodes to obtain opposite charge polarities. The opposite charge polarities will then cause the therapeutic agent to ionize. The ionized therapeutic agent will then be driven into the wound and/or the tissue surrounding the wound as result of the repulsive force between the treatment and return electrodes. Where the therapeutic agent comprises silver, for example, activation of the controller can cause ionized silver ions to migrate into the bed sore(s) of the subject and thereby kill any bacteria present therein.

In another, the therapeutic agent can be motivated into the wound and/or the tissue surrounding the wound via dielectrophoresis. Dielectrophoresis involves providing a non-uniform AC or DC electric field to a compound or therapeutic agent. The non-uniform electric field, in addition to inducing a dipole in the compound or agent, sets up an electrical field gradient that provides an electromotive force on the newly polarized compound or agent, the magnitude and direction of which are dependent on several factors. A more detailed explanation of dielectrophoresis and its operating principles are disclosed in U.S. patent application Ser. Nos. 11/874,859 (hereinafter, “the '859 application”) and Ser. No. 13/107,582, the entirety of each of which is hereby incorporated by reference.

To deliver the therapeutic agent via dielectrophoresis, the electrode of the system can comprise an interdigitated electrode. In general, an interdigitated electrode can include any set of at least two electrodes that contain interwoven projections. For example, the interdigitated electrode can be comprised of a first electrically-conductive member that is separated by an insulator from a second electrically-conductive member. Each of the first and second electrically-conductive members can comprise a “comb” electrode (i.e., an electrode having a number of relatively long, flat prongs that are evenly spaced) whose prongs are interleaved with one another. The interdigitated electrode can additionally include at least one passage sufficient to allow at least one therapeutic agent to pass therethrough. More specific details concerning the design and function of interdigitated electrodes are disclosed in the '859 application.

The controller can be activated to send an AC signal having certain characteristics to the interdigitated electrode. The controller can be activated to cycle through at least one decade of frequencies ranging from about 0.1 Hz to about 20,000 Hz. For example, an AC signal can have an orienting frequency of about 0.1 Hz to about 100 Hz, a motivating frequency of between about 100 Hz and about 20,000 Hz, and an amplitude of between about 1 V to about 10 V. Additionally, an AC signal can be applied for between about 1 minute and about 30 minutes. A more specific description of the electrical signal and the logic used to modulate the electrical signal is disclosed in the '859 application. The system can include multiple electrode channels and the controller can be configured or programmed to turn all the electrode channels off before measuring the current one channel at a time, then after all measurements are made, the controller can turn on all the electrode at the voltage determined. For example, the off time of all the channels can be less than about two seconds and the current can be measured about every minute. If the controller turns the voltage to a maximum set value and the current is less than 3 uA current, for example, then the controller can be programmed to no longer deliver current at that voltage.

Application of the electrical signal motivates the therapeutic agent into the wound and/or the tissue surrounding the wound. For example, application of an AC signal to the interdigitated electrode provides a non-uniform electric field that induces a dipole on the at least one therapeutic agent. This, in turn, sets up an electrical field gradient that provides an electromotive force on the newly polarized therapeutic agent to drive the agent into the wound and/or the tissue surrounding the wound.

Either before, simultaneous with, or subsequent to delivery of the therapeutic agent, fluid or debris can be removed from the wound at step 126. In particular, a vacuum can be activated to suction fluid and/or debris from the wound at step 126. For example, with reference to FIG. 6, vacuum 98 can be activated so that negative pressure is created within porous material of the wound dressing. As shown in FIG. 6, the negative pressure or suction causes fluid (e.g., blood, puss, etc.) and/or debris (e.g., bacteria, fungi, scab fragments, cellular debris, etc.) to be pulled through porous material of the wound dressing (as schematically illustrated by the arrows pointing away from wound 11B), and through fluid line 101 into a waste reservoir (not shown).

It is known that operation of conventional electromotive devices (e.g., iontophoresis and dielectrophoresis devices) can create unwanted localized pH alterations at the wound site due to accumulation of electrolysis products, cellular necrosis, and build-up of dead tissue. Such alterations can shield microorganisms, such as bacteria and fungi from delivery of the therapeutic agents and thereby contribute to further wound development. By removing unwanted fluid and/or debris during treatment, an optimal healing environment is created by method 120. Additionally, continuous delivery the therapeutic agent into the wound or tissue surrounding the wound along with application of negative pressure or suction advantageously creates a flow pattern (collectively indicated by arrows in FIG. 6) about the wound so that the therapeutic agent can flow all over the wound bed. By manipulating the amount of negative pressure and the flow rate of the therapeutic agent(s), the flow pattern created about the wound site can be selectively controlled for optimal tissue perfusion and treatment efficiency.

As indicated at step 128 of method 120, steps 124 and 126 can be optionally repeated until wound 11B is sufficiently healed. It will be appreciated that other medical instruments or apparatus can be used to supplement one or more steps of method 120, depending upon the particular type of wound 11B being treated.

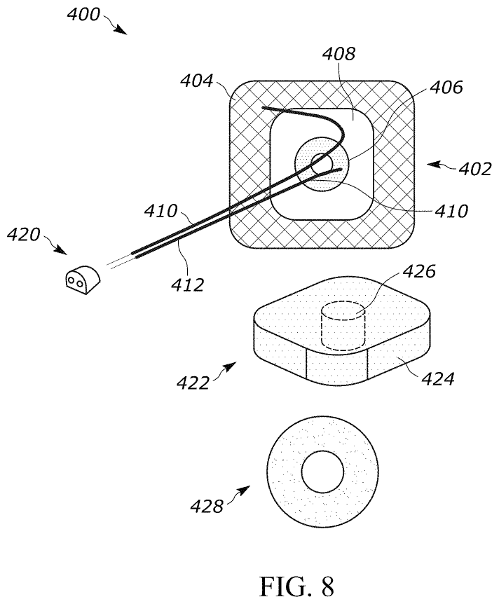

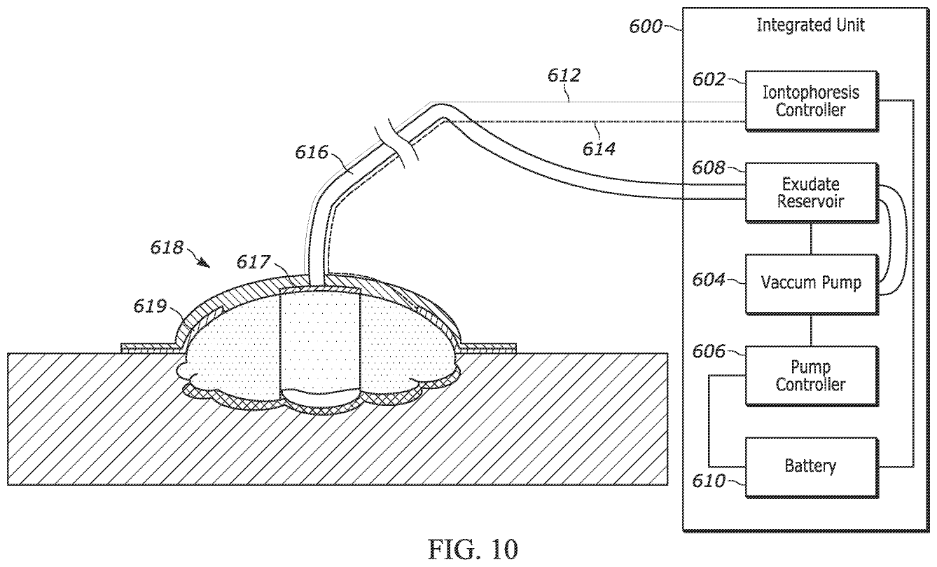

Referring to FIG. 8, in an embodiment, a system 400 for healing a wound bed surrounded by skin of a subject in need thereof is provided. System 400 can comprise an occlusive drape 402 having an outer region 404 and an inner region 406 separated by an insulated region 408. The outer region can be configured to be in direct or indirect contact with the skin of the subject. System 400 can further include an insulated first electrical wire 410 in electrical communication with the outer region of the occlusive drape and an insulated second electrical wire 412 in electrical communication with the inner region of the occlusive drape. The first insulated electrical wire and the second insulated electrical wire have opposing polarities. In certain aspects, the first electrical wire is a cathode and the second electrical wire is an anode. The system can further include an integrated device (shown in FIG. 10) comprising a vacuum configured to generate negative pressure and a controller configured to control activation and iontophoresis parameters of the first insulated electrical wire and the second insulated electrical wire. The system can also include connector 420 having a vacuum port attachable to the vacuum of the integrated device and a controller port attachable to the controller of the integrated device. The system can further include a foam 422 having a non-conductive region 424 surrounding a conductive core 426. The conductive core can be configured to be in contact with the second inner region of the occlusive drape and configured to be in contact with the wound bed. The conductive core can comprise an ionic therapeutic agent, such as a positively charged therapeutic agent. In certain aspects, the system can further include a conductive hydrocolloid dressing 428 having a top surface configured to contact the first outer region of the occlusive drape and a bottom surface configured to contact the skin of the subject.

FIG. 10 illustrates an exemplary integrated device comprising a vacuum configured to generate negative pressure and a controller configured to control activation and iontophoresis parameters of the first insulated electrical wire and the second insulated electrical wire. FIG. 10 is illustrated with respect to the wound dressing depicted in FIG. 9 but could be used with other would dressings as disclosed herein. Integrated device/unit 600 includes an iontophoresis controller 602; a vacuum pump 604 that is in communication with a vacuum pump controller 606; and a power source 610, such as a battery, that is in communication with both vacuum pump controller 606 and iontophoresis controller 602 although separate power sources could be used to provide power to each component. As shown in FIG. 10, an anode wire 612 is in electrical communication with controller 602 and anode 617 in wound dressing 618. A cathode wire 614 is in electrical communication with controller 602 and cathode 619 in wound dressing 618. Of course, the polarities of the electrodes could be reversed. To apply negative pressure, vacuum pump 604 is activated to apply negative pressure to wound dressing 618, removing any exudate which exits through drain 616 to exudate reservoir 608, which, in this embodiment, is part of integrated device 600.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance.

What is claimed is:

1. A system for healing a wound of a subject in need thereof, the system comprising:
a wound dressing comprising:
an upper layer comprising a porous material covered by an outer film defining a vacuum port;
a lower tissue-contacting layer;
a therapeutic agent disposed in the wound dressing; and
at least one electrode or at least one capacitor disposed in the wound dressing and in electrical communication with the therapeutic agent;
a controller in electrical communication with the at least one electrode or the at least one capacitor;
a vacuum configured to generate negative pressure within the porous material; and
a fluid line having one end connected to the vacuum port and another end connected to the vacuum.

2. The system of claim 1, wherein the therapeutic agent is ionic form.

3. The system of claim 1, wherein the lower tissue-contacting layer contains the therapeutic agent in ionic form and wherein the at least one electrode overlies the lower tissue-contacting layer.

4. The method of claim 1, wherein the wound dressing further comprises an intermediate layer between the upper layer and the lower tissue-contacting layer.

5. The system of claim 4, wherein the lower tissue-contacting layer contains the therapeutic agent and the upper layer or the intermediate layer comprises the at least one electrode.

6. The system of claim 4, wherein the intermediate layer comprises a porous dielectric barrier layer overlying the lower tissue-contacting layer.

7. The system of claim 4, wherein the at least one electrode comprises a first and a second electrode having opposing polarities and wherein:
the lower tissue-contacting layer comprises the therapeutic agent;
the intermediate layer comprises the first electrode and overlies the tissue-contacting layer; and
the outer film comprises the second electrode.

8. The system of claim 1, wherein the at least one electrode is an electrically conductive polymer foam.

9. The system of claim 1, further comprising an electroactive polymer disposed in the wound dressing that expands when an electrical stimulus is applied thereto.

10. The system of claim 4, wherein the intermediate layer comprises a plurality of segments, the plurality of segments comprising a first segment and a second segment of electrically conductive polymer foam separated by a segment of dielectric foam, the first and second segments having opposing polarities, the plurality of segments creating the at least one capacitor.

11. The system of claim 1, wherein the at least one capacitor is a plurality of capacitors.

12. The system of claim 1, further comprising a sensor configured to sense a physiological parameter associated with the wound and generate a sensor signal based on the physiological parameter.

13. The system of claim 12, wherein the controller is in communication with the sensor and programmed to generate a drive signal to automatically control activation of the at least one electrode or parameters of the electrical field delivered by the at least one electrode to heal the subject's wound in response to the sensor signal.

14. The system of claim 12, wherein the sensor is a temperature, pH, blood oxygen saturation level, pressure, light, or impedance sensor.

15. The system of claim 12, wherein the sensor is disposed in the wound dressing.

16. The system of claim 1, further comprising an ultrasound emitter, a light emitter, a cold plasma emitter, or a shock wave emitter disposed in the wound dressing or in a component in communication with the wound dressing.

17. The system of claim 16, further comprising a dormant sensor disposed in the wound dressing that is activated by the respective ultrasound emitter, light emitter, cold plasma emitter, or shock wave emitter.

18. A system for healing a wound bed surrounded by skin of a subject in need thereof, the system comprising:
an occlusive drape having an outer region and an inner region separated by an insulated region, the outer region configured to be in direct or indirect contact with the skin of the subject;
an insulated first electrical wire in electrical communication with the outer region of the occlusive drape;
an insulated second electrical wire in electrical communication with the inner region of the occlusive drape, the first insulated electrical wire and the second insulated electrical wire having opposing polarities;
an integrated device comprising a vacuum configured to generate negative pressure and a controller configured to control activation and iontophoresis parameters of the first insulated electrical wire and the second insulated electrical wire;
a connector having a vacuum port attachable to the vacuum of the integrated device and a controller port attachable to the controller of the integrated device; and
a foam having a non-conductive region surrounding a conductive core, the conductive core configured to be in contact with the second inner region of the occlusive drape and configured to be in contact with the wound bed.

19. The system of claim 18, wherein the conductive core comprises an ionic therapeutic agent.

20. The system of claim 19, wherein the first electrical wire is a cathode, the second electrical wire is an anode, and the ionic therapeutic agent is positively charged.

21. The system of claim 18, further comprising a conductive hydrocolloid dressing having an top surface configured to contact the first outer region of the occlusive drape and bottom surface configured to contact the skin of the subject.
